(19) United States
(12) Patent Application Publication
(10) Pub. No.: US 2018/0008206 A1
(43) Pub. Date: Jan. 11, 2018

(54) SYSTEMS AND METHODS TO DETERMINE SURROGATES OF BLOOD PRESSURE

(71) Applicant: Cardiac Pacemakers, Inc., St. Paul, MN (US)

(72) Inventors: Jeffrey E. Stahmann, Ramsey, MN (US); Arthur J. Foster, Blaine, MN (US); Bin Mi, Plymouth, MN (US); Michael J. Kane, St. Paul, MN (US); Pramodsingh H. Thakur, Woodbury, MN (US); William J. Linder, Golden Valley, MN (US); Qi An, Blaine, MN (US); Keith R. Maile, New Brighton, MN (US)

(21) Appl. No.: 15/643,112

(22) Filed: Jul. 6, 2017

Related U.S. Application Data
(60) Provisional application No. 62/359,636, filed on Jul. 7, 2016.

(51) Int. Cl. A61B 5/00; A61N 1/375; A61B 7/04; A61B 7/02; A61B 5/0205; A61B 5/0452; A61N 1/39; A61B 5/11; A61B 5/053; A61B 5/0215; A61B 5/021

(52) U.S. Cl. CPC A61B 5/7275; A61N 1/3956; A61N 1/3925; A61N 1/3756; A61B 7/04; A61B 7/023; A61B 5/0059; A61B 5/11; A61B 5/0205; A61B 5/0452; A61B 5/0215; A61B 5/02125; A61B 5/053

(57) ABSTRACT

Embodiments of the present disclosure relate to systems and methods for determining a subject's blood pressure using one or more implantable medical devices (IMDs). In an embodiment, a medical system comprises: at least one implantable medical device configured to sense signals associated with heart sounds of a subject and a processing unit communicatively coupled to the at least one implantable medical device. The processing unit is configured to: receive heart sound signals corresponding to the signals associated with the heart sounds; and calculate a surrogate of the subject's blood pressure using at least one heart sound signal of the received heart sound signals.

### CROSS-REFERENCE TO RELATED APPLICATION

This application claims priority to Provisional Application No. 62/359,636, filed Jul. 7, 2016, which is herein incorporated by reference in its entirety.

### TECHNICAL FIELD

Embodiments of the present disclosure relate to medical devices and systems for sensing physiological parameters. More specifically, embodiments of the disclosure relate to systems and methods for determining a subject's blood pressure using one or more implantable medical devices (IMDs).

### BACKGROUND

High and low blood pressure can lead to a number of health problems. For example, high blood pressure can lead to heart disease, heart attack and stroke, while low blood pressure can lead to dizziness, syncope and nausea. As such, it can be important to monitor a person's blood pressure. However, current systems and methods to measure a person's blood pressure, such as using a cuff, are usually transient in time. Accordingly, there is a need in the art for alternative systems and methods for determining a person's blood pressure over a longer period of time.

### SUMMARY

Embodiments of the disclosure relate to systems and methods for determining a subject's blood pressure using one or more implantable medical devices (IMDs). Example embodiments include the following.

In an Example 1, a medical system comprises: at least one implantable medical device configured to sense signals associated with heart sounds of a subject; a processing unit communicatively coupled to the at least one implantable medical device, wherein the processing unit is configured to: receive heart sound signals corresponding to the signals associated with the heart sounds; and calculate a surrogate of the subject's blood pressure using at least one heart sound signal of the received heart sound signals.

In an Example 2, the medical system of Example 1, wherein the at least one implantable medical device senses signals associated with heart sounds of a subject using at least one of: an acoustic sensor and a motion sensor.

In an Example 3, the medical system of Example 1, wherein the processing unit is configured to calculate the surrogate of the subject's blood pressure by calculating an amplitude of the at least one heart sound signal corresponding to an S1 heart sound of the subject.

In an Example 4, the medical system of Example 3, wherein the processing unit is configured to determine the subject's blood pressure based on the calculated surrogate by applying, to the amplitude of the at least one heart sound signal, at least one of a scaling factor, an exponential factor and an offset factor.

In an Example 5, the medical system of any of Examples 1-4, wherein the at least one heart sound signal comprises a plurality of heart sound signals, wherein at least two of the plurality of heart sound signals correspond to different types of heart sounds.

In an Example 6, the medical system of Example 5, wherein the processing unit is configured to calculate the surrogate of the subject's blood pressure by calculating a ratio of an amplitude of a heart sound signal corresponding to an S1 heart sound and an amplitude of a heart sound signal corresponding to an S2 heart sound.

In an Example 7, the medical system of Example 6, wherein the processing unit is configured to determine the subject's blood pressure based on the calculated surrogate by applying, to the ratio, at least one of a scaling factor, an exponential factor and an offset factor.

In an Example 8, the medical system of any of Examples 3 and 7, wherein the processing unit is further configured to adjust at least one of the scaling factor, the exponential factor and the offset factor, based on a result from a secondary blood pressure test.

In an Example 9, the medical system of any of Examples 1-8, wherein the at least one heart sound signal corresponds to at least one of: an S1 heart sound, an S2 heart sound, an M1 heart sound, a T1 heart sound, an A2 heart sound, and a P2 heart sound.

In an Example 10, a method comprises: sensing, by at least one implantable medical device, at least one physiological parameter associated with a subject's circulatory system; determining a plurality of timings associated with the at least one sensed physiological parameter; and calculating a surrogate of the subject's blood pressure using the determined plurality of timings.

In an Example 11, the method of Example 10, wherein sensing, by at least one implantable medical device, at least one physiological parameter comprises: sensing a fiducial of a QRS complex of the subject by the at least one implantable medical device; sensing a heart sound of the subject by the at least one implantable medical device; wherein determining a plurality of timings comprises determining a timing of the sensed component and determining a timing of the sensed heart sound; and wherein calculating the surrogate comprises calculating a difference between the timing of the sensed component and the timing of the sensed heart sound.

In an Example 12, the method of Example 11, wherein a first implantable medical device of the at least one implantable medical device senses the component and a second implantable medical device of the at least one implantable medical device senses the heart sound, wherein the first implantable medical device is different than the second implantable medical device, and wherein the heart sound is an S2 heart sound.

In an Example 13, the method of any of Examples 10-12, wherein sensing, by at least one implantable medical device, the at least one physiological parameter comprises: sensing a fiducial of a QRS complex of the subject by the at least one implantable medical device; sensing a pulsation of an artery of the subject by the at least one implantable medical device; wherein determining a plurality of timings comprises determining a timing the fiducial of the QRS complex and determining a timing of the sensed pulsation of the artery; and wherein calculating the surrogate comprises calculating a pulse transit time based on the timing of the sensed fiducial and the timing of the sensed pulsation of the artery.

In an Example 14, the method of Example 12, wherein the at least one implantable medical device senses the pulsation of an artery using at least one of: an impedance sensor, a motion sensor, a pressure sensor and an optical sensor.

In an Example 15, the method of any of Examples 10-14, wherein sensing, by at least one implantable medical device, the plurality of timings comprises: sensing a first pulsation of an artery of the subject by a first implantable medical device of the at least one implantable medical device, wherein the first pulsation is sensed at a first location of the artery; sensing a second pulsation of the artery by a second implantable medical device of the at least one implantable medical device, wherein the second pulsation is sensed at a second location of the artery that is different than the first location and wherein the first implantable medical device is different than the second implantable medical device; wherein determining the plurality of timings comprises determining a timing of the first pulsation of the artery and determining a timing of the second pulsation of the artery; and wherein calculating the surrogate of the subject's blood pressure using the plurality of timings comprises calculating a pulse transit time based on the first timing and the second timing.

In an Example 16, a medical system comprises: at least one implantable medical device configured to sense signals associated with heart sounds of a subject; a processing unit communicatively coupled to the at least one implantable medical device, wherein the processing unit is configured to: receive heart sound signals corresponding to the signals associated with the heart sounds; and calculate a surrogate of the subject's blood pressure using at least one heart sound signal of the received heart sound signals.

In an Example 17, the medical system of Example 16, wherein the at least one implantable medical device senses signals associated with heart sounds of a subject using at least one of an acoustic sensor and a motion sensor.

In an Example 18, the medical system of Example 16, wherein the processing unit is configured to calculate the surrogate of the subject's blood pressure by calculating an amplitude of the at least one heart sound, the at least one heart sound signal corresponding to an S1 heart sound of the subject.

In an Example 19, the medical system of Example 18, wherein the processing unit is configured to determine the subject's blood pressure based on the calculated surrogate by applying, to the amplitude of the at least one heart sound signal, at least one of a scaling factor, an exponential factor and an offset factor.

In an Example 20, the medical system of Example 19, wherein the processing unit is further configured to adjust at least one of the scaling factor, the exponential factor and the offset factor based on a result from a secondary blood pressure test.

In an Example 21 ,the medical system of Example 16, wherein the at least one heart sound signal comprises a plurality of heart sound signals, wherein at least two of the plurality of heart sound signals correspond to different types of heart sounds.

In an Example 22, the medical system of Example 21, wherein the processing unit is configured to calculate the surrogate of the subject's blood pressure by calculating a ratio of an amplitude of a heart sound signal corresponding to an S1 heart sound and an amplitude of a heart sound signal corresponding to an S2 heart sound.

In an Example 23, the medical system of Example 22, wherein the processing unit is configured to determine the subject's blood pressure based on the calculated surrogate by applying, to the ratio, at least one of a scaling factor, an exponential factor and an offset factor.

In an Example 24, the medical system of Example 23, wherein the processing unit is further configured to adjust at least one of the scaling factor, the exponential factor and the offset factor, based on a result from a secondary blood pressure test.

In an Example 25, the medical system of Example 16, wherein the at least one heart sound signal corresponds to at least one of: an S1 heart sound, an S2 heart sound, an M1 heart sound, a T1 heart sound, an A2 heart sound, and a P2 heart sound.

In an Example 26, the medical system of Example 16, wherein the at least one implantable medical device is at least one of: an implantable cardioverter defibrillator, a subcutaneous implantable cardioverter defibrillator, a leadless implantable cardioverter defibrillator and an implantable cardiac monitor.

In an Example 27, the medical system of Example 16, wherein the processing device is located external to the implantable medical device.

In an Example 28, a medical system comprises: at least one implantable medical device configured to sense a plurality of physiological parameters associated with a subject's circulatory system; a processing unit communicatively coupled to the at least one implantable medical device, wherein the processing unit is configured to: receive a plurality of signals corresponding to the plurality of sensed physiological parameters; determine a plurality of timings associated with the received signals; and calculate a surrogate of the subject's blood pressure using the determined plurality of timings.

In an Example 29, the medical system of Example 28, wherein the at least one implantable medical device is configured to sense at least one physiological parameter by sensing a fiducial of a QRS complex of the subject and sensing a heart sound of the subject; wherein the received signals include a signal corresponding to the sensed fiducial and a signal corresponding to the sensed heart sound; wherein the processing unit is configured to determine a plurality of timings by determining a timing of the sensed fiducial from the received signal corresponding to the sensed fiducial and determining a timing of the sensed heart sound from the received signal corresponding to the sensed heart sound; and wherein the processing unit is configured to calculate the surrogate by calculating a difference between the timing of the sensed fiducial and the timing of the sensed heart sound.

In an Example 30, the medical system of Example 29, wherein a first implantable medical device of the at least one implantable medical device senses the fiducial of the QRS complex and a second implantable medical device of the at least one implantable medical device senses the heart sound, wherein the first implantable medical device is different than the second implantable medical device, and wherein the heart sound is an S2 heart sound.

In an Example 31, the medical system of Example 28, wherein the at least one implantable medical device is configured to sense at least one physiological parameter by sensing a fiducial of a QRS complex of the subject and sensing a pulsation of an artery of the subject; wherein the received signals include a signal corresponding to the sensed fiducial and a signal corresponding to the sensed pulsation of the artery; wherein the processing unit is configured to determine a plurality of timings by determining a timing of the sensed fiducial from the received signal corresponding to the sensed fiducial and determining a timing of the sensed pulsation of the artery from the received signal corresponding to the sensed pulsation of the artery; and wherein the processing unit is configured to calculate the surrogate by calculating a pulse transit time based on the timing of the sensed fiducial and the timing of the sensed pulsation of the artery.

In an Example 32, the medical system of Example 31, wherein the at least one implantable medical device senses the pulsation of an artery using at least one of: an impedance sensor, a motion sensor, a pressure sensor and an optical sensor.

In an Example 33, the medical system of Example 28, wherein the at least one implantable medical device is configured to sense at least one physiological parameter by sensing a first pulsation of an artery of the subject at a first location of the artery and by sensing a second pulsation of the artery at a second location of the artery, wherein the first location is different than the second location; wherein the received signals include a signal corresponding to the first sensed pulsation and a signal corresponding to the second sensed pulsation; wherein the processing unit is configured to determine a plurality of timings by determining a first timing of the first sensed pulsation from the received signal corresponding to the first sensed pulsation and determining a second timing of the second pulsation of the artery from the received signal corresponding to the second sensed pulsation; and wherein the processing unit is configured to calculate the surrogate by calculating a pulse transit time based on the first timing and the second timing.

In an Example 34, a method comprises: sensing, by at least one implantable medical device, at least one physiological parameter associated with a subject's circulatory system; calculating a surrogate of the subject's blood pressure based on the sensed at least one physiological parameter, wherein the at least one physiological parameter is a heart sound or a timing associated with a cardiac parameter.

In an Example 35, the method of Example 34, further comprising determining the subject's blood pressure based on the calculated surrogate, wherein determining the subject's blood pressure based on the calculated surrogate comprises applying, to the calculated surrogate, at least one of a scaling factor and an offset factor.

While multiple embodiments are disclosed, still other embodiments of the present disclosure will become apparent to those skilled in the art from the following detailed description, which shows and describes illustrative embodiments of the disclosed subject matter. Accordingly, the drawings and detailed description are to be regarded as illustrative in nature and not restrictive.

### BRIEF DESCRIPTION OF THE DRAWINGS

FIG. 1 is a schematic illustration of a system including an IMD for determining a subject's blood pressure, in accordance with embodiments of the present disclosure.

FIG. 2 is a block diagram depicting an illustrative medical system for determining a subject's blood pressure, in accordance with embodiments of the present disclosure.

FIG. 3 is a flow diagram depicting an illustrative process for determining a subject's blood pressure, in accordance with embodiments of the present disclosure.

While the disclosed subject matter is amenable to various modifications and alternative forms, specific embodiments have been shown by way of example in the drawings and are described in detail below. The intention, however, is not to limit the disclosed subject matter to the particular embodiments described. On the contrary, the disclosed subject matter is intended to cover all modifications, equivalents, and alternatives falling within the scope of the disclosed subject matter as defined by the appended claims.

As the terms are used herein with respect to ranges of measurements (such as those disclosed immediately above), “about” and “approximately” may be used, interchangeably, to refer to a measurement that includes the stated measurement and that also includes any measurements that are reasonably close to the stated measurement, but that may differ by a reasonably small amount such as will be understood, and readily ascertained, by individuals having ordinary skill in the relevant arts to be attributable to measurement error, differences in measurement and/or manufacturing equipment calibration, human error in reading and/or setting measurements, adjustments made to optimize performance and/or structural parameters in view of differences in measurements associated with other components, particular implementation scenarios, imprecise adjustment and/or manipulation of objects by a person or machine, and/or the like.

Although the term “block” may be used herein to connote different elements illustratively employed, the term should not be interpreted as implying any requirement of, or particular order among or between, various blocks disclosed herein. Similarly, although illustrative methods may be represented by one or more drawings (e.g., flow diagrams, communication flows, etc.), the drawings should not be interpreted as implying any requirement of, or particular order among or between, various steps disclosed herein. However, certain embodiments may require certain steps and/or certain orders between certain steps, as may be explicitly described herein and/or as may be understood from the nature of the steps themselves (e.g., the performance of some steps may depend on the outcome of a previous step). Additionally, a “set,” “subset,” or “group” of items (e.g., inputs, algorithms, data values, etc.) may include one or more items, and, similarly, a subset or subgroup of items may include one or more items. A “plurality” means more than one.

### DETAILED DESCRIPTION

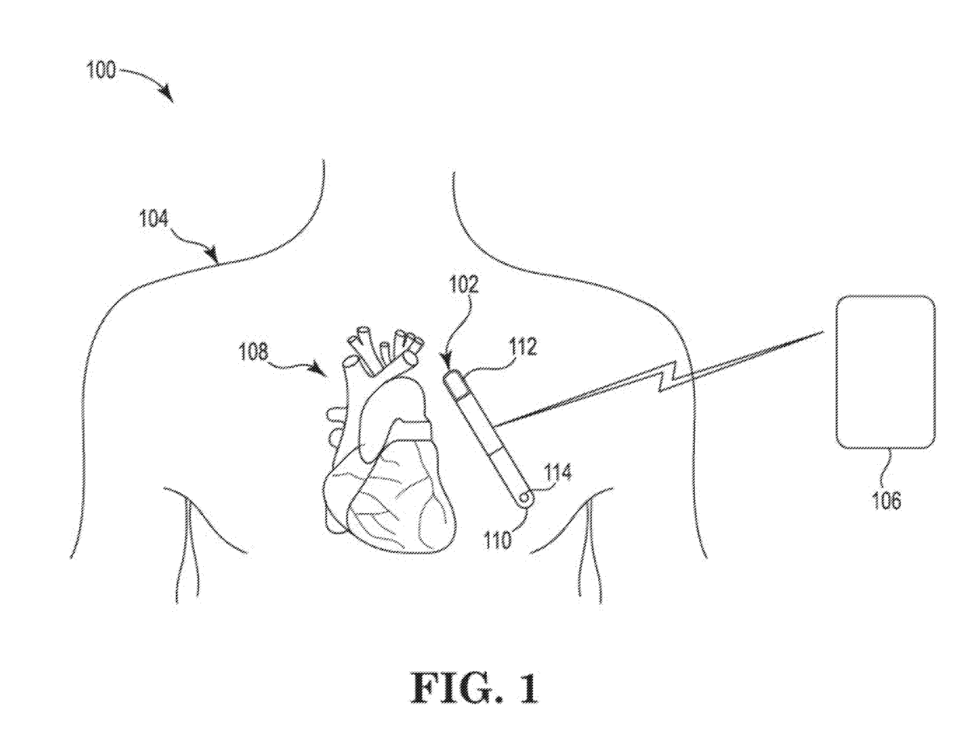

FIG. 1 is a schematic illustration of a system 100 including an implantable medical device (IMD) 102 implanted within a subject's body 104 and configured to be communicatively coupled to a processing unit 106. The system 100 may be used to monitor (e.g., sense and/or record) physiological parameters, determine surrogates based on the monitored physiological parameters, diagnose, and/or provide therapy in accordance with embodiments of the disclosure.

In embodiments, the IMD 102 may be implanted subcutaneously within an implantation location or pocket in the subject's chest or abdomen and may be configured to monitor (e.g., sense and/or record) physiological parameters associated with the subject's circulatory system including, for example, the physiological parameters associated with the subject's heart 108. In embodiments, the IMD 102 may be an implantable cardiac monitor (ICM) (e.g., an implantable diagnostic monitor (IDM), an implantable loop recorder (ILR), etc.) configured to monitor physiological parameters such as, for example, the subject's cardiac activation signals, heart sounds, pulsations of arteries, oxygen saturations, and/or the like.

In embodiments, the IMD 102 may be configured to monitor physiological parameters that may include one or more signals indicative of a subject's physical activity level and/or metabolic level, such as an acceleration signal. In embodiments, the IMD 102 may be configured to monitor physiological parameters associated with one or more other organs, systems, and/or the like.

For example, the IMD 102 may include sensors or circuitry for detecting cardiac system signals, circulatory system signals, respiratory system signals, and/or signals related to subject activity. In embodiments, the IMD 102 may be configured to sense intrathoracic impedance, from which various respiratory parameters may be derived, including, for example, respiratory tidal volume and minute ventilation. In embodiments, the IMD 102 may be configured to sense cardiac impedance, from which various cardiac parameters may be derived, including, for example, left and right ventricular activity. Sensors and associated circuitry may be incorporated in connection with the IMD 102 for detecting one or more body movement or body posture and/or position related signals. For example, accelerometers and/or GPS devices may be employed to detect subject activity, subject location, body orientation, and/or torso position.

For purposes of illustration, and not of limitation, various embodiments of devices that may be used to monitor physiological parameters in accordance with the present disclosure are described herein in the context of IMDs that may be implanted under the skin in the chest region of a subject. In embodiments, however, the IMD 102 may include any type of IMD, any number of different components of an implantable system, and/or the like having a housing and being configured to be implanted in a subject's body 104. For example, the IMD 102 may include a control device, a monitoring device, a pacemaker, an implantable cardioverter defibrillator (ICD), a subcutaneous implantable cardioverter defibrillator (S-ICD), a leadless implantable cardioverter defibrillator (L-ICD), a cardiac resynchronization therapy (CRT) device, a neural stimulation device, and/or the like, and may be an implantable medical device known in the art or later developed, for providing therapy and/or diagnostic data about the subject's body and/or the IMD 102. In various embodiments, the IMD 102 may include both defibrillation and pacing/CRT capabilities (e.g., a CRT-D device).

The IMD 102 may be configured to sense and/or record at regular intervals, continuously, and/or in response to a detected event. In embodiments, such a detected event may be detected by one or more sensors of the IMD 102, another IMD (not shown), an external device (not shown), and/or the like. In addition, the IMD 102 may be configured to detect a variety of physiological signals that may be used in connection with various diagnostic, therapeutic, and/or monitoring implementations.

As shown, the IMD 102 may include a housing 110 having two electrodes 112 and 114 coupled thereto. According to embodiments, the IMD 102 may include any number of electrodes (and/or other types of sensors such as, e.g., sound sensors, pressure sensors, impedance sensors, optical sensors, thermometers, barometers, motion or impact sensors (e.g., accelerometers, inertial measuring units (IMUs)), and/or the like) in any number of various types of configurations, and the housing 110 may include any number of different shapes, sizes, and/or features. In embodiments, the IMD 102 may be configured to sense physiological parameters and record the physiological parameters. For example, the IMD 102 may be configured to activate (e.g., periodically, continuously, upon detection of an event, and/or the like), record a specified amount of data (e.g., physiological parameters) in a memory, and communicate that recorded data to a processing unit 106. In the case of an IMD, for example, the IMD 102 may activate, record cardiac signals for a certain period of time, deactivate, and activate to communicate the recorded signals to the processing unit 106.

In embodiments, the processing unit 106 may be incorporated into the IMD 102 or external to the IMD 102. For example, in embodiments where the processing unit 106 is external to the IMD 102, the processing unit 106 may be incorporate into another IMD (not shown). Alternatively, in embodiments where the processing unit 106 is external to the IMD 102, the processing unit 106 may be positioned on the subject, near the subject, or in any location external to the subject.

In embodiments, the IMD 102 and the processing unit 106 may communicate through a wired and/or wireless communication link. For example, the IMD 102 and the processing unit 106 may be coupled through a short-range radio link, such as Bluetooth, IEEE 802.11, and/or a proprietary wireless protocol. The term “communication link” may refer to an ability to communicate some type of information in at least one direction between at least two devices, and should not be understood to be limited to a direct, persistent, or otherwise limited communication channel. That is, according to embodiments, the communication link may be a persistent communication link, an intermittent communication link, an ad-hoc communication link, and/or the like. The communications link may facilitate uni-directional and/or bi-directional communication between the IMD 102 and the processing unit 106. Data and/or control signals may be transmitted between the IMD 102 and the processing unit 106 to coordinate the functions of the IMD 102 and/or the processing unit 106. In embodiments, subject data may be downloaded from one or more of the IMD 102 and the processing unit 106 periodically or on command. The physician and/or the subject may communicate with the IMD 102 and the processing unit 106, for example, to acquire subject data or to initiate, terminate, or modify recording and/or therapy.

The illustrative system 100 shown in FIG. 1 is not intended to suggest any limitation as to the scope of use or functionality of embodiments of the subject matter disclosed throughout this disclosure. Neither should the illustrative system 100 be interpreted as having any dependency or requirement related to any single component or combination of components illustrated in FIG. 1. For example, in embodiments, the illustrative system 100 may include additional components. Additionally, any one or more of the components depicted in FIG. 1 can be, in embodiments, integrated with various ones of the other components depicted therein (and/or components not illustrated). Any number of other components or combinations of components can be integrated with the illustrative system 100 depicted in FIG. 1, all of which are considered to be within the ambit of this disclosure.

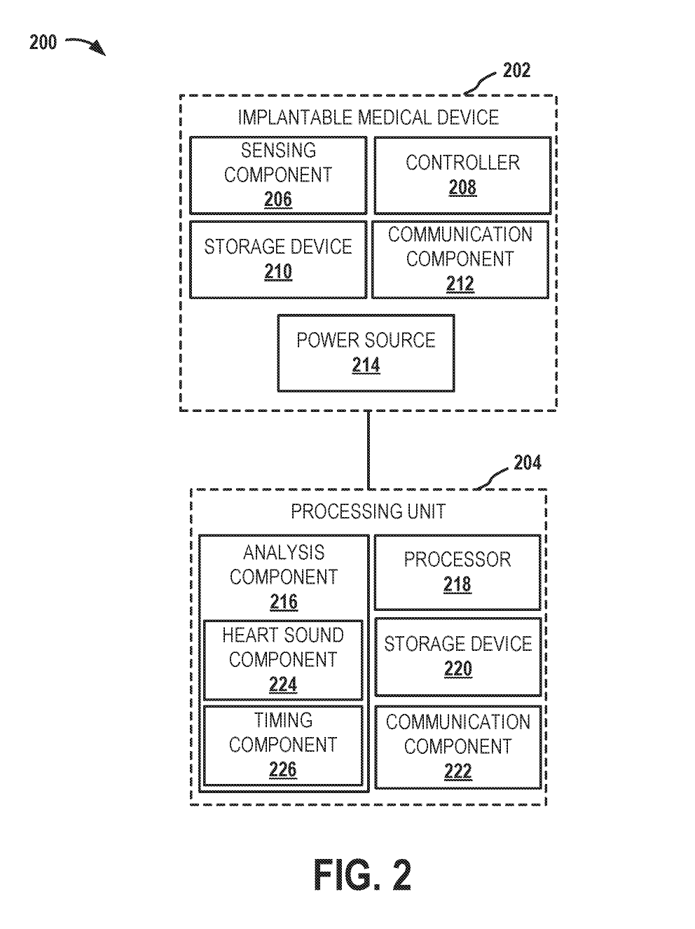

FIG. 2 is a block diagram depicting an illustrative medical system 200 for determining a subject's blood pressure, in accordance with embodiments of the present disclosure. As shown, the system 200 includes an implantable medical device (IMD) 202 and a processing unit 204. Embodiments of the system may include more than one IMD 202 and/or more than one processing unit 204. The IMD 202 may be, be similar, to, include, or be included in, the IMD 102 depicted in FIG. 1; and the processing unit 204 may be, be similar to, include, or be included in, the processing unit 106 depicted in FIG. 1. While the processing unit 204 is depicted as located external to the IMD 202, in embodiments, the processing unit 204 may be incorporated into the IMD 202. Alternatively, the processing unit 204 may be incorporated into a different IMD (not shown). Alternatively, the processing unit 204 may be located external to a subject. In embodiments, the processing unit 204 may be distributed between multiple devices. That is, for example, the processing unit 204 may refer to a number of different processing devices and/or virtual processors, each disposed on (and/or instantiated by) an IMD or an external device.

According to embodiments illustrated in FIG. 2, the IMD 202 includes a sensing component 206, a controller 208, a storage device 210, a communication component 212, and a power source 214. The IMD 202 may sense physiological parameter signals using the sensing component 206 that may include, for example, one or more electrodes (not shown), one or more sensors (not shown), or a combination thereof. In embodiments, the sensing component 206 may include any number of electrical circuits, electronic components, processing units, program components and/or the like.

In embodiments, the sensing component 206 may sense intrinsic cardiac electrical signals, for example, QRS complexes and/or fiducials of QRS complexes, in a manner similar to known electrocardiogram (ECG) electrodes. In embodiments, the sensing component 206 may be configured to sense other subject physiologic or environmental parameters in addition to, or alternative to, cardiac signals. In embodiments, the sensing component 206 may include temperature sensors (e.g., thermocouples or thermistors), barometers, acoustic sensors, pressure sensors, impedance sensors, optical sensors, motion or impact sensors (e.g., accelerometers, inertial measuring units (IMUs)), strain sensors, Doppler systems, ultrasound sensors, and/or the like, in any number of various types of configurations. The foregoing sensors allow the IMD 202 to be capable of sensing and recording physiologic parameters such as, for example, heart sounds, pulsations of arteries, oxygen saturations, subject movement, posture, respiratory cycles and/or the like.

As stated above, the sensing component 206 may be configured to sense heart sounds using one or more acoustic sensors. Additionally or alternatively sensing component 206 may be configured to sense the motion associated with closure of cardiac valves using a motion sensor (e.g. an accelerometer). In embodiments, motion-based detection of cardiac valve closure is, in effect, a way to detect heart sounds. Examples of heart sounds the sensing component 206 may be configured to sense include one or more of the following: the first heart sound (S1), the second heart sound (S2) and components thereof. That is, in embodiments, the sensing component 206 may be configured to monitor the S1 heart sound, which occurs at the onset of ventricular contraction, and the components thereof which include the closure of the mitral valve (M1) and the tricuspid valve (T1). Additionally or alternatively, in embodiments, the sensing component 206 may be configured to monitor the S2 heart sound, which indicates the end of the ventricular systole and the beginning of the ventricular relaxation, and the components thereof, which include the closure of the aortic valve (A2) and the closure of the pulmonary valve (M2). In embodiments, the sensing component 206 may record a time that one or more heart sounds are sensed.

Additionally or alternatively, the sensing component 206 may be configured to sense QRS complexes and/or fiducials of QRS complexes in a manner similar to known electrocardiogram (ECG) electrodes. In embodiments, the sensing component 206 may record times that one or more of the Q waves, R waves, S waves and/or other fiducials of the QRS complex are sensed.

Additionally or alternatively, the sensing component 206 may be configured to sense cardiac impedance indicative of the subject's right and/or left ventricle contracting. In embodiments, the sensing component 206 may record times of one or more of these changes in impedances.

Additionally or alternatively, the sensing component 206 may be configured to sense pulsations of arteries using, for example, an impedance sensor, a motion sensor, a pressure sensor and an optical sensor. For example, in embodiments, the IMD may be placed proximate to an artery of the subject. As a pulse of blood passes by an impedance sensor, a motion sensor or a pressure sensor of the sensing component 206, the impedance sensor, the motion sensor or the pressure sensor may register a decrease or increase in impedance, motion or pressure, respectively. Additionally or alternatively, as another example, as a pulse of blood passes by an optical sensor of the sensing component 206, the reflection of the light emitted by the optical sensor may change, which may indicate a pulsation of the artery. In embodiments, the sensing component 206 may include more than one sensor configured to sense pulsations of arteries. For example, the sensing component 206 may include two or more impedance sensors, two or more motion sensors, two or more pressure sensors, two or more optical sensors or a combination thereof. In embodiments where the sensing component 206 includes more than one sensor, the more than one sensor may sense a pulsation at the same location on the artery or different locations of the artery. Additionally or alternatively, the sensing component 206 may be incorporated into two different IMDs 202 and each IMD 202 may sense a pulsation of the artery at respective locations. In embodiments, however, the pulsation measured at a first location of the respective locations may be the same pulse of blood passing through the artery as the pulsation measured at a second location of the respective locations. As such, a pulse transit time (PTT) may be calculated by the processing device 204, as described below.

In embodiments, the sensing component 206 may measure a combination of the heart sounds, QRS complexes, fiducials of the QRS complexes and pulsations of arteries. Additionally or alternatively, more than one IMD 202 may sense the heart sounds, QRS complexes, fiducials of the QRS complexes, pulsations of the arteries and/or a combination thereof. Sensed signals associated with heart sounds, QRS complexes, fiducials of the QRS complexes and/or pulsations of arteries may transmitted by a communication component 212 of the IMD 202 to the processing unit 204, where the processing unit 204 determines one or more surrogates for a subject's blood pressure based on the received signals, as described below.

The controller 208 may include, for example, a processor and/or the like. The controller 208 may be any arrangement of electronic circuits, electronic components, processors, program components and/or the like configured to store and/or execute programming instructions, to direct the operation of the other functional components of the IMD 202, for example, execute the instructions of the sensing component 206, and may be implemented, for example, in the form of any combination of hardware, software, and/or firmware.

The storage device 210 may be used to store information sensed by the IMD 202 according to some implementations. The storage device 210 may include volatile and/or non-volatile memory, and may store instructions that, when executed by the IMD 202 cause methods and processes to be performed by the IMD 202. In embodiments, the controller 208 may process instructions and/or data stored in the storage device 210 control sensing operations performed by the IMD 202, to control communications performed by the IMD 202, and/or the like.

The communication component 212 may include, for example, circuits, program components, and one or more transmitters and/or receivers for communicating wired or wirelessly with one or more other devices such as, for example, the processing unit 204. According to various embodiments, the communication component 212 may include one or more transmitters, receivers, transceivers, transducers, and/or the like, and may be configured to facilitate any number of different types of wireless communication such as, for example, radio-frequency (RF) communication, microwave communication, infrared communication, acoustic communication, inductive communication, conductive communication, and/or the like. The communication component 212 may include any combination of hardware, software, and/or firmware configured to facilitate establishing, maintaining, and using any number of communication links. In embodiments, the communication component 212 of the medical device 202 facilitates wired or wireless communication with the processing unit 204. In embodiments, the communication component 212 may also facilitate communications with other medical devices such as, for example, to facilitate coordinated operations between the medical devices.

In other embodiments, other forms of wired or wireless telemetry may be utilized for communications. For example, in embodiments, other RF telemetry technologies may be employed. Alternatively, and/or additionally, inductive telemetry, acoustic telemetry and/or the like may be employed for communicating with, e.g., the processing unit 204. In embodiments, conductive telemetry may be employed, in which case, for example, the communication component 212 may interact with one or more sensing/therapy electrode(s) to transmit and/or receive communications encoded in electrical pulses.

The power source 214 provides electrical power to the other operative components (e.g., the sensing component 206, the storage device 210, and the communication component 212) of the IMD 202, and may be any type of power source suitable for providing the desired performance and/or longevity requirements of the IMD 202. In various embodiments, the power source 214 may include one or more batteries, which may be rechargeable (e.g., using an external energy source). The power source 214 may include one or more capacitors, energy conversion mechanisms, and/or the like. Power sources for medical devices such as the medical device 202 are well known, and are therefore not discussed in greater detail herein.

As shown in FIG. 2, the processing unit 204 includes an analysis component 216, a processor 218, a storage device 220, and a communication component 222. In embodiments, the analysis component 216 may be implemented in any combination of hardware, software, and/or firmware, and may be implemented, at least in part, by the processor 218. In embodiments, the analysis component 216 may include a heart sound component 224 and/or a timing component 226. The heart sound component 224 and/or timing component 226 may determine a subject's blood pressure based on the received signals corresponding to sensed signals associated with the heart sounds, QRS complexes and/or pulsations of the arteries sensed by the IMD 202.

In embodiments, the heart sound component 224 may determine a subject's blood pressure based on one or more of the signals associated with heart sounds of a subject. That is, one or more heart sounds of a subject have been found to be correlated to a subject's blood pressure. As such, a surrogate of the subject's blood pressure may be calculated by the sound component 224 based on sensed signals associated with a subject's heart sounds. For example, the S1 heart sound of a subject has been found to be positively correlated with a correlation coefficient of approximately 0.785 to a subject's systolic blood pressure. By receiving a signal corresponding to a subject's S1 heart sound, the heart sound component 224 may calculate a surrogate of the subject's blood pressure by determining an amplitude of the S1 heart sound. As another example, the S1/S2 heart sound ratio of a subject has been found to be positively correlated with a correlation coefficient of approximately 0.914 to a subject's systolic blood pressure. As such, by receiving a signal corresponding to a subject's S1 and S2 heart sounds, the heart sound component 224 may calculate a surrogate of the subject's blood pressure by calculating a ratio of the S1 heart sound amplitude with respect to the S2 heart sound amplitude. As even another example, the S2 heart sound of a subject has been found to be positively correlated with a correlation coefficient of approximately 0.745 to a subject's average aortic blood pressure. As such, by receiving a signal corresponding to a subject's S2 heart sound, the heart sound component 224 may calculate a surrogate of the subject's aortic blood pressure by calculating an amplitude of the S2 heart sound. As other examples, additionally or alternatively, one or more components of the S1 and S2 heart sounds (i.e., the M1, T1 and A2, P2, respectively) may also be used to calculate one or more surrogates for a subject's blood pressure.

Additionally or alternatively, the timing component 226 may determine a subject's blood pressure based on signals corresponding to one or more timings of one or more physiological parameters. That is, for example, similar to heart sounds, one or more pulse transit times (PTT) for a subject has been found to be positively correlated to a subject's blood pressure. The PTT of a subject is the amount of time it takes the pulse pressure waveform, generated by a beat of the heart, to propagate through a length of the arterial tree. To determine a PTT, one or more timings of one or more physiological parameters may be used. For example, a first physiological parameter may be sensed at a first location, a second physiological parameter may be sensed at a second location, and the two sensed parameters, along with the time between them may be used to calculate a PTT. In embodiments, the first physiological parameter may be a heart sound (e.g., an S1 heart sound), a fiducial of a QRS complex (e.g., an R wave, Q wave and/or S wave) and/or the like. The second physiological parameter may be sensed perfusion (e.g., sensed using impedance plethysmography), a blood pressure pulse, and/or the like.

For example, the time between a sensed R wave of a QRS complex and a sensed pulsation of an artery can be used to calculate a PTT. In embodiments, the distance between the heart and the location on the artery where the pulsation is sensed may be used to adjust the PTT. That is, for example, the time between the sensed R wave and the sensed pulsation of the artery may be divided by the distance between the heart and the location on the artery where the pulsation is sensed. By receiving a signal corresponding to a timing of an R wave for a subject and a signal corresponding to a timing of a pulsation of the subject's artery, the timing component 226 may calculate a PTT for the subject. In embodiments, the timing of the sound of a heart murmur and/or a change of cardiac impedance indicative of the left ventricle contracting may be used in place of or in conjunction with the timing of the R wave. As another example, a pulsation of an artery sensed at two different locations on the artery may also be used to determine a PTT for the subject. These are only examples, however, and not meant to be limiting. Instead, other methods used to calculate the PTT of a subject may be used and are embodied herein.

Additionally or alternatively, in embodiments, the timing component 226 may determine a subject's blood pressure based on signals corresponding to one or more timings of one or more physiological parameters that are negatively correlated with a subject's blood pressure. For example, the time between an R wave of a QRS complex and the S2 heart sound has been found to be negatively correlated with correlation coefficients of approximately −0.690 and approximately −0.740 to a subject's systolic blood pressure and diastolic blood pressure, respectively. That is, by receiving a signal corresponding to a time of a sensed R wave for a subject and a signal corresponding to a time of a sensed S2 heart sound, the timing component 226 may calculate a surrogate for the subject's blood pressure based on the time difference.

In embodiments, after a surrogate (e.g., S1, S1/S2, PTT and/or time between R-S2) for a subject's blood pressure is calculated, the timing component 226 may perform one or more operations on the surrogate to determine the subject's blood pressure. In embodiments, performing one or more operations on the surrogate may include one or more of: applying a scaling factor to the surrogate, applying an exponential factor to the surrogate and/or applying an offset factor to the surrogate. That is, an example equation may be x*S^{r} +/−O, wherein x is the scaling factor that may be any real number, S is the surrogate, r is an exponential factor that may be any real positive number and O is the offset factor that may be any real number.

In embodiments, the scaling factor, exponential factor and/or the offset factor may be adjusted based on secondary blood pressure test, for example, a cuff test. That is, for example, if a scaling factor, exponential factor, and/or an offset factor is determining a subject's blood pressure to be higher or lower than a secondary blood pressure test indicates, the scaling factor, the exponential factor and/or offset factor may be adjusted either up or down based on the results of the secondary blood pressure test.

Additionally or alternatively, since the surrogate may not have a 1-to-1 or a −1-to-1 correlation to a subject's blood pressure, a margin of error may be included in the determination of the subject's blood pressure. For example, a margin of error of +/−5%, 10%, 15% and/or the like may be included with the determination of a subject's blood pressure. That is, for example, if an amplitude of an S1 heart sound is determined to be 200 sound milli-pressure level (smpl), and a scaling factor is determined to be 0.60, the subject's blood pressure might be determined to be 120, which is the product of the scaling factor and the amplitude of the S1 heart sound. However, in embodiments, the determined blood pressure of 120 may include a +/−5%, 10%, 15% and/or the like margin of error to compensate for any inexactness in the correlation between the amplitude of a subject's S1 heart sound and a subject's blood pressure.

In embodiments, a pressure sensor capable of measuring atmospheric pressure, e.g. an atmospheric pressure sensor external to the body, can be used to adjust the blood pressure determined by system 200. In embodiments, the atmospheric pressure may be measured by a component (not shown) that is incorporated into the processing unit 204. In embodiments, a heart sound, QRS complex, fiducial of a QRS complex and/or pulsation may be sensed by the sensing component 206 and an atmospheric pressure measurement may be acquired at approximately the same time and at the same location. In embodiments, the ratio of blood pressure measurement to atmospheric pressure may be determined. This ratio may then be used at other times and locations to adjust the blood pressure measurement to changes in measured atmospheric pressure.

In embodiments, one or more of the embodiments described above for determining a subject's blood pressure may be used as a validation check against the other embodiments for determining a subject's blood pressure.

As stated above, the processing unit 204 includes a processor 218 that may be, be identical to, be similar to, include, or be included within, the controller 208 of the IMD 202. In embodiments, the processor 218 may be a programmable micro-controller or microprocessor, and/or may include one or more programmable logic devices (PLDs) and/or application specific integrated circuits (ASICs). In some implementations, the processor 218 may include memory. The processor 218 may include digital-to-analog (D/A) converters, analog-to-digital (A/D) converters, timers, counters, filters, switches, and/or the like. The processor 218 may execute instructions and perform desired tasks as specified by the instructions.

The processor 218 may also be configured to store information in the storage device 220 and/or access information from the storage device 220. The storage device 220 may include volatile and/or non-volatile memory, and may store instructions that, when executed by the processor 218 cause methods and processes to be performed by the processing unit 204. In embodiments, the processor 218 may process instructions and/or data stored in the storage device 220 to determine a surrogate of a subject's blood pressure using, for example, the heart sound component 224 and/or the timing component 226, to control communications performed by the processing unit 204, and/or the like.

The storage device 220 may be, be identical to, be similar to, include, or be included within the storage device 210. That is, for example, the storage device 220 may include volatile and/or non-volatile memory, and may store instructions that, when executed by the processor 218 cause methods and processes to be performed by the processing unit 204. In embodiments, the processor 218 may process instructions and/or data stored in the storage device 220 to control communications performed by the communication component 222.

The communication component 222 may communicate with a variety of devices described in detail above. For example, if the processing unit's 204 analysis component 220 determines a blood pressure for a subject, the communication component 222 may output a visual display of the determined blood pressure to a display device (not shown), including by not limited to, a display device located on a wearable item.

The illustrative system 200 shown in FIG. 2 is not intended to suggest any limitation as to the scope of use or functionality of embodiments of the present disclosure. Neither should the illustrative system 200 be interpreted as having any dependency or requirement related to any single component or combination of components illustrated therein. Additionally, various components depicted in FIG. 2 may be, in embodiments, integrated with various ones of the other components depicted therein (and/or components not illustrated), all of which are considered to be within the ambit of the present disclosure.

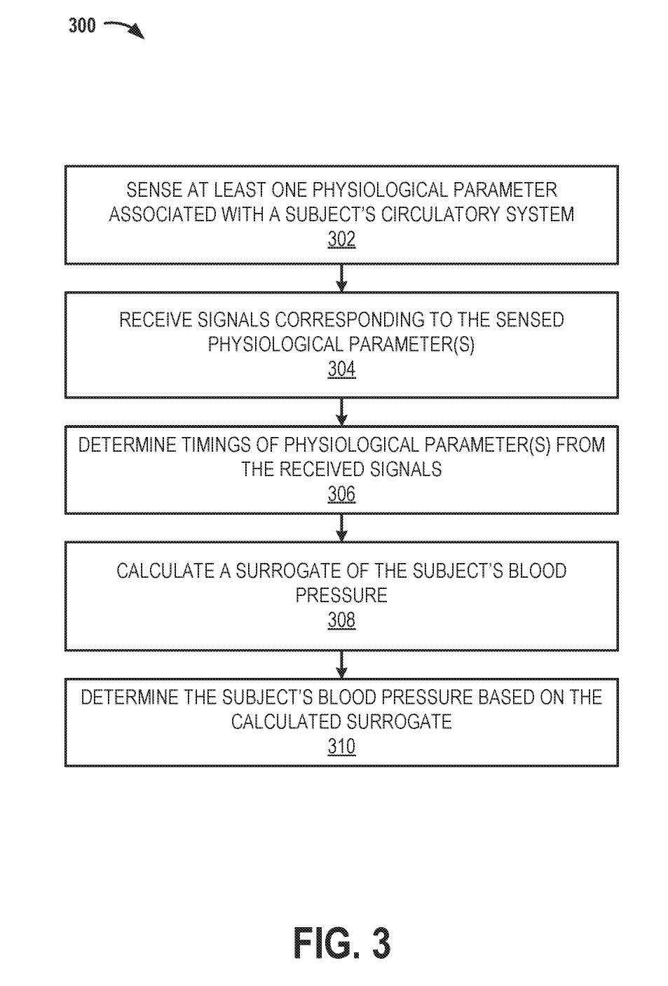

FIG. 3 is a flow diagram depicting an illustrative method 300 for determining a subject's blood pressure, in accordance with embodiments of the present disclosure. In embodiments, the method 300 includes sensing at least one physiological parameter associated with a subject's circulatory system (block 302). In embodiments, the at least one physiological parameter may be, be similar to, include, or be included in the same physiological parameters that are described above in relation to FIGS. 1 and 2. For example, the sensed physiological parameters may include, but are not limited to, the subject's cardiac activation signals, heart sounds, pulsations of arteries, oxygen saturations, impedance, and/or the like. In embodiments, the cardiac activation signals may include sensing QRS complexes and/or fiducials of QRS complexes of the subject using ECG electrodes. In embodiments, the heart sounds may include sensing one or more of the following heart sounds: S1, S2, Ml, T1, A2 and/or M2 using, e.g., an acoustic sensor and/or an motion sensor (e.g., an accelerometer). In embodiments, the pulsations of arteries may include sensing a pulsation at a location on an artery and/or the same pulsation at different locations on the artery using acoustic sensors, optical sensor or a combination thereof.

In embodiments, one or more IMDs may be used to sense the at least physiological parameter. In embodiments, the one or more IMDs may be, be similar to, include, or be included in the IMD 102 depicted in FIG. 1 and/or the IMD 202 depicted in FIG. 2. For example, the IMD may be an ICM, IDM, ILR, ICD, S-ICD, L-ICD, CRT, CRT-D, neural stimulation device and/or a combination thereof.

In embodiments, the method 300 may include receiving signals corresponding to the at least one physiological parameter (block 304). In embodiments, an IMD may send the signals corresponding to the at least one physiological parameter to a processing unit via one or more communication components. In embodiments, the timings associated with the sensed signals may be sent to a processing unit as part of the same signal or via a different signal. In embodiments, the processing unit may be, be similar to, include, or be included in the processing unit 106 depicted in FIG. 1 and/or the processing unit 204 depicted in FIG. 2.

In embodiments, the method 300 may include determining timings of one or more of the at least one physiological parameter based on the received signals (method 306). The timings may be, be similar to, include, or be included the timings discussed above in relation to FIG. 2. For example, the timings of the physiological parameters may be the timings of one or more heart sounds including, but not limited to, the timings of the S1, S2, M1, T2, A2 and/or M2 heart sound. Additionally or alternatively, the timings may be the timings of one or more pulsations of arteries and/or the timings of a single pulse of blood passing through an artery that is measured at two different locations of the artery.

In embodiments, the method 300 may include calculating a surrogate of the subject's blood pressure (block 308). In embodiments, the calculated surrogate of the subject's blood pressure may be, be similar to, include, or be included the calculated surrogates of the subject's blood pressure discussed above in relation to FIG. 2. For example, one or more surrogates of the subject's blood pressure may be an amplitude of a subject's S1 heart sound, a ratio of the amplitudes of a subject's S1 heart sound in relation to a subject's S2 heart sound, a PTT and/or the time between a sensed R wave and a sensed S2 hear sound.

In embodiments, the method 300 includes determining a subject's blood pressure based on the calculated surrogate (block 310). In embodiments, determining a subject's blood pressure based on the calculated surrogate may be, be similar to, include, or be included the determining a subject's blood pressure based on the calculated surrogate discussed above in relation to FIG. 2. For example, a scaling factor, an exponential factor and/or an offset factor may be applied to the calculated surrogate. Additionally or alternatively, the scaling factor, the exponential factor and/or the offset factor may be adjusted up or down based on results from a secondary blood pressure test.

Using various embodiments described herein, a subject's blood pressure may be monitored over a longer period of time than many conventional methods allow. As such, acute and/or chronic changes in blood pressure may be better managed.

Various modifications and additions can be made to the exemplary embodiments discussed without departing from the scope of the present disclosure. For example, while the embodiments described above refer to particular features, the scope of this disclosure also includes embodiments having different combinations of features and embodiments that do not include all of the described features. Accordingly, the scope of the present disclosure is intended to embrace all such alternatives, modifications, and variations as fall within the scope of the claims, together with all equivalents thereof.

What is claimed is:

1. A medical system comprising:
at least one implantable medical device configured to sense signals associated with heart sounds of a subject;
a processing unit communicatively coupled to the at least one implantable medical device, wherein the processing unit is configured to:
receive heart sound signals corresponding to the signals associated with the heart sounds; and
calculate a surrogate of the subject's blood pressure using at least one heart sound signal of the received heart sound signals.

2. The medical system of claim 1, wherein the at least one implantable medical device senses signals associated with heart sounds of a subject using at least one of an acoustic sensor and a motion sensor.

3. The medical system of claim 1, wherein the processing unit is configured to calculate the surrogate of the subject's blood pressure by calculating an amplitude of the at least one heart sound, the at least one heart sound signal corresponding to an S1 heart sound of the subject.

4. The medical system of claim 3, wherein the processing unit is configured to determine the subject's blood pressure based on the calculated surrogate by applying, to the amplitude of the at least one heart sound signal, at least one of a scaling factor, an exponential factor and an offset factor.

5. The medical system of claim 4, wherein the processing unit is further configured to adjust at least one of the scaling factor, the exponential factor and the offset factor based on a result from a secondary blood pressure test.

6. The medical system of claim 1, wherein the at least one heart sound signal comprises a plurality of heart sound signals, wherein at least two of the plurality of heart sound signals correspond to different types of heart sounds.

7. The medical system of claim 6, wherein the processing unit is configured to calculate the surrogate of the subject's blood pressure by calculating a ratio of an amplitude of a heart sound signal corresponding to an S1 heart sound and an amplitude of a heart sound signal corresponding to an S2 heart sound.

8. The medical system of claim 7, wherein the processing unit is configured to determine the subject's blood pressure based on the calculated surrogate by applying, to the ratio, at least one of a scaling factor, an exponential factor and an offset factor.

9. The medical system of claim 8, wherein the processing unit is further configured to adjust at least one of the scaling factor, the exponential factor and the offset factor, based on a result from a secondary blood pressure test.

10. The medical system of claim 1, wherein the at least one heart sound signal corresponds to at least one of: an S1 heart sound, an S2 heart sound, an M1 heart sound, a T1 heart sound, an A2 heart sound, and a P2 heart sound.

11. The medical system of claim 1, wherein the at least one implantable medical device is at least one of: an implantable cardioverter defibrillator, a subcutaneous implantable cardioverter defibrillator, a leadless implantable cardioverter defibrillator and an implantable cardiac monitor.

12. The medical system of claim 1, wherein the processing device is located external to the implantable medical device.

13. A medical system comprising:
at least one implantable medical device configured to sense a plurality of physiological parameters associated with a subject's circulatory system;
a processing unit communicatively coupled to the at least one implantable medical device, wherein the processing unit is configured to:
receive a plurality of signals corresponding to the plurality of sensed physiological parameters;
determine a plurality of timings associated with the received signals; and
calculate a surrogate of the subject's blood pressure using the determined plurality of timings.

14. The medical system of claim 13, wherein
the at least one implantable medical device is configured to sense at least one physiological parameter by sensing a fiducial of a QRS complex of the subject and sensing a heart sound of the subject;
wherein the received signals include a signal corresponding to the sensed fiducial and a signal corresponding to the sensed heart sound;
wherein the processing unit is configured to determine a plurality of timings by determining a timing of the sensed fiducial from the received signal corresponding to the sensed fiducial and determining a timing of the sensed heart sound from the received signal corresponding to the sensed heart sound; and
wherein the processing unit is configured to calculate the surrogate by calculating a difference between the timing of the sensed fiducial and the timing of the sensed heart sound.

15. The medical system of claim 14, wherein a first implantable medical device of the at least one implantable medical device senses the fiducial of the QRS complex and a second implantable medical device of the at least one implantable medical device senses the heart sound, wherein the first implantable medical device is different than the second implantable medical device, and wherein the heart sound is an S2 heart sound.

16. The medical system of claim 13, wherein
the at least one implantable medical device is configured to sense at least one physiological parameter by sensing a fiducial of a QRS of the subject and sensing a pulsation of an artery of the subject;
wherein the received signals include a signal corresponding to the sensed fiducial and a signal corresponding to the sensed pulsation of the artery;
wherein the processing unit is configured to determine a plurality of timings by determining a timing of the sensed fiducial from the received signal corresponding to the sensed fiducial and determining a timing of the sensed pulsation of the artery from the received signal corresponding to the sensed pulsation of the artery; and
wherein the processing unit is configured to calculate the surrogate by calculating a pulse transit time based on the timing of the sensed fiducial and the timing of the sensed pulsation of the artery.

17. The medical system of claim 16, wherein the at least one implantable medical device senses the pulsation of an artery using at least one of: an impedance sensor, a motion sensor, a pressure sensor and an optical sensor.

18. The medical system of claim 13, wherein
the at least one implantable medical device is configured to sense at least one physiological parameter by sensing a first pulsation of an artery of the subject at a first location of the artery and by sensing a second pulsation of the artery at a second location of the artery, wherein the first location is different than the second location;
wherein the received signals include a signal corresponding to the first sensed pulsation and a signal corresponding to the second sensed pulsation;
wherein the processing unit is configured to determine a plurality of timings by determining a first timing of the first sensed pulsation from the received signal corresponding to the first sensed pulsation and determining a second timing of the second pulsation of the artery from the received signal corresponding to the second sensed pulsation; and
wherein the processing unit is configured to calculate the surrogate by calculating a pulse transit time based on the first timing and the second timing.

19. A method comprising:
sensing, by at least one implantable medical device, at least one physiological parameter associated with a subject's circulatory system; and
calculating a surrogate of the subject's blood pressure based on the sensed at least one physiological parameter, wherein the at least one physiological parameter is a heart sound or a timing associated with a cardiac parameter.

20. The method of claim 19, further comprising determining the subject's blood pressure based on the calculated surrogate, wherein determining the subject's blood pressure based on the calculated surrogate comprises applying, to the calculated surrogate, at least one of a scaling factor, an exponential factor and an offset factor.
